(19) United States
(12) Patent Application Publication
(10) Pub. No.: US 2003/0135065 A1
(43) Pub. Date: Jul. 17, 2003

(54) Process for the production of L-DOPA ethyl ester

(76) Inventors: Ramy Lidor-Hadas, Kfar Saba (IL); Anton Frenkel, Modiin (IL); Eliezer Bahar, Tel Aviv (IL); Gisan Atili, Tira (IL)

Correspondence Address: Cooper & Dunham LLP, 1185 Avenue of the Americas, New York, NY, 10036, US

(21) Appl. No.: 10/292,053

(22) Filed: Nov. 12, 2002

Related U.S. Application Data
(60) Provisional application No. 60/350,705, filed on Nov. 13, 2001.

(51) Int. Cl. C07C 229/36

(52) U.S. Cl. 560/39

(57) ABSTRACT

A process for manufacturing a highly purified, stable, non-hygroscopic, crystalline composition of L-DOPA ethyl ester. The L-DOPA ethyl ester is an active ingredient in pharmaceutical preparations for the treatment of patients suffering from Parkinson's Disease and related indications.

This application claims benefit of U.S. Provisional Application No. 60/350,705, filed Nov. 13, 2001, the contents of which are hereby incorporated by reference.

Throughout this application, various references are identified by authors and full citation. Disclosure of these publications in their entireties are hereby incorporated by reference into this application to more fully describe the state of the art to which this invention pertains.

### FIELD OF THE INVENTION

The present invention relates to a process for manufacturing a highly purified, stable, non-hygroscopic, crystalline composition of L-DOPA ethyl ester. L-DOPA ethyl ester (also known as LDEE) is used as an active ingredient in pharmaceutical preparations for the treatment of patients suffering from Parkinson's disease (PD), and related indications.

### BACKGROUND OF THE INVENTION

Typically, Parkinsonian patients are routinely treated with a combination of levodopa (L-DOPA) and a DOPA decarboxylase inhibitor such as carbidopa or benserazide. Unfortunately, after an initial period of satisfactory, smooth and stable clinical benefit from L-DOPA therapy lasting on the average 2-5 years, the condition of many patients deteriorates and they develop complex dose-related as well as unpredictable response fluctuations. The causes of the response fluctuations are probably multiple and complex, but pharmacokinetic problems (primarily faulty absorption of L-DOPA) may play a critical role. There is a correlation between the clinical fluctuations and the oscillations of L-DOPA plasma levels. Many of the problems are a result of the unfavorable pharmacokinetic properties of L-DOPA, i.e., very poor solubility, poor bio-availability and short half-life in vivo.

A more suitable drug for therapy of PD would be the L-DOPA ethyl ester. However, it has been difficult to develop the L-DOPA ethyl ester in a form suitable for pharmaceutical use:

In view of the potential toxicity that might arise from methanol formation the ethyl ester would ideally have been most suitable for assessment in humans. However, the ethyl ester could not be crystallized as its hydrochloride salt because of its hygroscopic potential. The methyl ester was therefore developed for use in humans.

(Stocci, F. et al., Movement Disorders, 7:249-256, (1992); at 254).

L-DOPA ethyl ester is described in the literature as the hydrochloride salt. However, it is difficult to isolate as a crystalline salt and therefore was described as an amorphous solid (Fix, et al., Pharm. Research 6(6):501-505 (1989)) which is not suitable for pharmaceutical use. Cooper, et al., Clinical Neuropharmacology 7:88-89 (1984) note that L-DOPA ethyl ester hydrochloride salt is hygroscopic and difficult to crystallize during synthesis. Clearly, a pure, stable, non-hygroscopic form of L-DOPA ethyl ester is needed for pharmaceutical purposes.

Salts and esters of L-DOPA, including the L-DOPA ethyl ester, are mentioned in Patent GB 1,342,286 for the treatment of alopecia. The only disclosure regarding the nature of the L-DOPA ethyl ester is that it can be prepared from L-DOPA by conventional methods. However, as noted above, preparation of L-DOPA ethyl ester by conventional methods yields a product which is not suitable for pharmaceutical use due to its impurity, its hygroscopicity, and its lack of stability.

Great Britain Patent No. 1,364,505 and corresponding U.S. Pat. No. 3,803,120, assigned to Hoffman-La Roche, describe the synthesis of L-DOPA ethyl ester hydrochloride salt and free base. This compound is used as an intermediate in the synthesis of other compounds and is not characterized in the patent specification. In agreement with the literature (Fix, et al., Pharm. Research 6(6):501-505 (1989); and Cooper, et al., Clin. Pharmacol. 7:88-89 (1984)) we have found that the L-DOPA ethyl ester hydrochloride salt synthesized by the methods described in these patents is hygroscopic, not stable, difficult to crystallize, and, as a result, difficult to purify. This material cannot be used for pharmaceutical compositions. Likewise, the L-DOPA ethyl ester free base as prepared in these two patents is impure and not stable and thus also is not suitable for pharmaceutical compositions. At best it can be used as a synthetic intermediate for further chemical synthesis as described in the cited patents.

Two references note the synthesis of racemic levodopa ethyl ester. (Ginssburg, et al., Zh. Obshch. Khim. 39:1168-1170 (1969) and Venter, et al., S. Afr. Tydskr. Chem. 31:135-137(1978)). Neither of these references prepare crystalline L-DOPA ethyl ester in a form suitable for pharmaceutical use and certainly there is no teaching or suggestion of the preparation of crystalline L-DOPA ethyl ester in a form suitable for pharmaceutical use. Both references prepare the material as an intermediate for the synthesis of other materials of interest. Recently, Milman et al. (U.S. Pat. No. 5,354,885) described a new process for preparing pharmaceutically acceptable, crystalline, non-hygroscopic L-DOPA ethyl ester as free base. The Milman process provides L-DOPA ethyl ester of high purity, wherein at least 97% by weight is the L-DOPA ethyl ester while L-DOPA, as an impurity, is present in less than 1% by weight of the composition.

The crystalline, non-hygroscopic L-DOPA ethyl ester composition produced according to the Milman process is highly stable and remains as at least 97% by weight L-DOPA ethyl ester after incubation for 6 months at 40° C. The availability of L-DOPA ethyl ester in such high purity made feasible the preparation of pharmaceutical compositions of L-DOPA ethyl ester, which compositions could not be successfully developed on a commercial scale until the development of the process.

The potential for increased demand of highly purified L-DOPA ethyl ester described in the U.S. Pat. No. 5,354,885, warrants research to find a simpler, more economical process for producing L-DOPA ethyl ester of high purity. While the Milman process produced a highly purified L-DOPA ethyl ester, the process is lengthy and complicated because it involves extraction steps.

The Milman process comprises reacting L-DOPA with ethanol in the presence of thionyl chloride or an acid catalyst to yield crude L-DOPA ethyl ester hydrochloride. Then volatiles are removed from the crude L-DOPA ethyl ester hydrochloride by vacuum distillation. The residue is then dissolved with water containing a suitable antioxidant and the pH is adjusted to between 6.0 and 7.0 using a suitable base to yield a solution containing L-DOPA ethyl ester free base. To obtain the free base in the solvent phase, the solution is extracted with a suitable solvent in the presence of a suitable antioxidant. The solvent phase is than concentrated at a temperature lower than 40° C. to form a precipitate. The precipitate is then recrystallized in the presence of a second suitable solvent containing a second suitable antioxidant to yield the composition of pharmaceutically acceptable, crystalline, non-hygroscopic L-DOPA ethyl ester free base.

More recently, U.S. Pat. No. 6,218,566 disclosed a process for manufacturing a composition comprising pharmaceutically acceptable, crystalline, non-hygroscopic L-DOPA ethyl ester as free base. The contents of U.S. Pat. No. 6,218,566, in their entirety, are hereby incorporated by reference to more completely describe the background of the invention.

### SUMMARY OF THE INVENTION

The subject invention provides a process for preparing a composition comprising pharmaceutically acceptable, crystalline, non-hygroscopic L-DOPA ethyl ester as free base, which process comprises:

(a) reacting L-DOPA with absolute ethanol to produce a nonaqueous solution of crude L-DOPA ethyl ester salt;

(b) removing residual volatiles, including ethanol, from the nonaqueous solution of step (a);

(c) adding toluene to the nonaqueous solution from step (b);

(d) treating the nonaqueous solution from step (c) to remove volatiles, including residual ethanol;

(e) adding a suitable base in water to the solution from step (d) under controlled conditions to precipitate a crude L-DOPA ethyl ester free base and to form an aqueous phase containing L-DOPA and an organic phase;

(f) separating the aqueous phase containing L-DOPA from step

(e) from the organic phase from step (e);

(g) collecting the precipitated crude L-DOPA ethyl ester free base from step (f);

(h) drying the precipitated crude L-DOPA ethyl ester free base collected in step (g); and

(i) recrystallizing the dried, precipitated crude L-DOPA ethyl ester free base from step (h) in the presence of a suitable solvent containing an antioxidant so as to produce the composition of pharmaceutically acceptable, crystalline, non-hygroscopic L-DOPA ethyl ester free base.

This invention also provides a process for preparing a composition comprising pharmaceutically acceptable, crystalline, non-hygroscopic L-DOPA ethyl ester as free base, which process comprises:

(a) reacting L-DOPA with absolute ethanol in the presence of thionyl chloride or an acid catalyst to produce a nonaqueous solution of crude L-DOPA ethyl ester salt;

(b) removing residual volatiles, including ethanol, from the solution of step (a);

(c) adding toluene to the nonaqueous solution from step (b);

(d) treating the nonaqueous solution from step (c) to remove volatiles, including residual ethanol;

(e) adding a suitable base in water to the solution from step (d) under controlled conditions to precipitate a crude L-DOPA ethyl ester free base and to form an aqueous phase containing L-DOPA and an organic phase;

(f) separating the aqueous phase containing L-DOPA from step (e) from the organic phase from step (e);

(g) adding toluene to the organic phase from step (f);

(h) collecting the precipitated L-DOPA ethyl ester free base from step (g);

(i) drying the precipitated crude L-DOPA ethyl ester free base collected in step (h); and

(j) recrystallizing the dried, precipitated crude L-DOPA ethyl ester free base from step (i) in the presence of a suitable solvent containing an antioxidant so as to produce the composition of pharmaceutically acceptable, crystalline, non-hygroscopic L-DOPA ethyl ester free base.

### DETAILED DESCRIPTION OF THE INVENTION

The subject invention provides a process for preparing a composition comprising pharmaceutically acceptable, crystalline, non-hygroscopic L-DOPA ethyl ester as free base, which process comprises:

(a) reacting L-DOPA with absolute ethanol to produce a nonaqueous solution of crude L-DOPA ethyl ester salt;

(b) removing residual volatiles, including ethanol, from the nonaqueous solution of step (a);

(c) adding toluene to the nonaqueous solution from step (b);

(d) treating the nonaqueous solution from step (c) to remove volatiles, including residual ethanol;

(e) adding a suitable base in water to the solution from step (d) under controlled conditions to precipitate a crude L-DOPA ethyl ester free base and to form an aqueous phase containing L-DOPA and an organic phase;

(f) separating the aqueous phase containing L-DOPA from step (e) from the organic phase from step (e);

(g) collecting the precipitated crude L-DOPA ethyl ester free base from step (f);

(h) drying the precipitated crude L-DOPA ethyl ester free base collected in step (g); and

(i) recrystallizing the dried, precipitated crude L-DOPA ethyl ester free base from step (h) in the presence of a suitable solvent containing an antioxidant so as to produce the composition of pharmaceutically acceptable, crystalline, non-hygroscopic L-DOPA ethyl ester free base.

In one embodiment, step (a) is performed in the presence of thionyl chloride, hydrogen chloride or toluene sulfonic acid.

In another embodiment, step (a) is performed in the presence of hydrogen chloride.

In a further embodiment, the crude L-DOPA ethyl ester salt produced in step (a) is L-DOPA ethyl ester hydrochloride.

In an additional embodiment, the removal of residual volatiles in step (b) is effected by vacuum distillation.

In yet another embodiment, the residual volatiles removed in step (b) are ethanol and excess HCl.

In one embodiment, the suitable base of step (e) is sodium hydroxide or ammonium hydroxide.

In an added embodiment, the suitable base of step (e) is sodium hydroxide.

In a further embodiment, the addition of the suitable base in step (e) effects an adjustment in the pH of the solution to a pH range between about 3.5 and about 5.5, preferably between 4.0 and 5.0, at which point an antioxidant may be added, and the adjustment of pH by the addition of suitable base continues to bring the pH of the solution to a pH range between about 7.6 and about 8.2 to precipitate a crude L-DOPA ethyl ester base.

In still another embodiment, the antioxidant of step (e) is ascorbic acid, sodium sulfite, sodium metabisulfite, propyl gallate, or vitamin E.

In one embodiment, the suitable antioxidant of step (e) is sodium metabisulfite.

In still another embodiment, the suitable base in step (e) has been previously cooled to a temperature less than room temperature.

In a further embodiment, the suitable base in step (e) has been previously cooled to a temperature from 0 to 3° C.

In an additional embodiment, step (e) further comprises the addition of sodium metabisulphite.

In yet another embodiment, the controlled conditions from step (e) are conditions in which addition of the base solution is slowly performed in a nitrogen atmosphere, and a trace amount of L-DOPA ethyl ester is added to induce formation of precipitate.

In an added embodiment, the method further comprises adding toluene to the organic phase of step (e).

In still another embodiment, the method further comprises adding toluene to the organic phase of step (f).

In one embodiment, the drying of step (h) is effected by azeotropic distillation.

In a further embodiment, the suitable solvent of step (i) is ethyl acetate, methylene chloride, or toluene.

In an additional embodiment, the suitable solvent of step (i) is ethyl acetate.

In an added the suitable antioxidant of step (i) is ascorbic acid, 2,6-Di-tert-butyl-4-methylphenol (BHT), butylated hydroxy anisol (BHA), propyl gallate, or vitamin E.

In a further embodiment, the antioxidant of step (i) is 2,6-Di-tert-butyl-4-methylphenol (BHT).

The subject invention provides a process for preparing a composition comprising pharmaceutically acceptable, crystalline, non-hygroscopic L-DOPA ethyl ester as free base, which process comprises:

(a) reacting L-DOPA with absolute ethanol in the presence of thionyl chloride or an acid catalyst to produce a nonaqueous solution of crude L-DOPA ethyl ester salt;

(b) removing residual volatiles, including ethanol, from the solution of step (a);

(c) adding toluene to the nonaqueous solution from step (b);

(d) treating the nonaqueous solution from step (c) to remove volatiles, including residual ethanol;

(e) adding a suitable base in water to the solution from step (d) under controlled conditions to precipitate a crude L-DOPA ethyl ester free base and to form an aqueous phase containing L-DOPA and an organic phase;

(f) separating the aqueous phase containing L-DOPA from step (e) from the organic phase from step (e);

(g) adding toluene to the organic phase from step (f);

(h) collecting the precipitated L-DOPA ethyl ester free base from step (g);

(i) drying the precipitated crude L-DOPA ethyl ester free base collected in step (h); and

(j) recrystallizing the dried, precipitated crude L-DOPA ethyl ester free base from step (i) in the presence of a suitable solvent containing an antioxidant so as to produce the composition of pharmaceutically acceptable, crystalline, non-hygroscopic L-DOPA ethyl ester free base.

The composition may comprise pharmaceutically acceptable, crystalline, non-hygroscopic L-DOPA ethyl ester as free base in an amount which is at least 95%, and preferably 97% and more preferably 98% by weight of the composition and L-DOPA in an amount which is less than 2% by weight of the composition.

This invention will be better understood from the Experimental Details which follow. However, one skilled in the art will readily appreciate that the specific methods and results discussed are merely illustrative of the invention as described more fully in the claims which follow thereafter.

Experimental Details

Description of the Process

A process for preparing a composition comprising pharmaceutically acceptable, crystalline, non-hygroscopic L-DOPA ethyl ester as free base in an amount which is at least 95%, and preferably 97% and more preferably 98% by weight of the composition and L-DOPA in an amount which is less than 2% by weight of the composition.

Synthesis of Crude L-DOPA Ethyl Ester

Absolute ethanol (395 g, 500 ml, 8.58 moles, 17 eq.) and L-Dopa (100 g, 0.507 moles, 1 eq.) were introduced into a 1 L reactor. The batch was cooled to 15° C. and HCl(g) (37.01 g, 1.014 mole, 2 eq.) was bubbled into the reaction mixture at 22±2° C. The reaction was heated to reflux (79° C.) and kept at reflux for 3 hours. The batch was then cooled to less than 30° C. and the reaction mixture was concentrated under vacuum for 1.5-2.5 hours to a volume of 190 ml (the distillate amount was 418 ml (332 g)). Distillation was performed under a vacuum of 80 mbar or less, and the jacket was not heated to higher than 55° C. At a vacuum of 80 mbar (60 mmHg), the distillation was started at Tᵢₙ=25° C. while the T_{jacket}=55° C. Toluene (100 ml) was added and the batch was cooled to less than 30° C. and concentrated to a volume of 190 ml by distillation under vacuum for 1-2 hours (the distillate amount was 100 ml). Distillation was performed under a vacuum of 80 mbar or less, and the jacket was not heated to higher than 55° C. At a vacuum of 80 mbar (60 mmHg), the distillation was started at Tᵢₙ=27° C. while the T_{jacket}=50° C. At the end of the distillation, the jacket of the reactor was cooled to 20° C. When the temperature in the reaction mixture reached 25° C., a mixture of 240 ml water and 37 g 5N NaOH previously cooled to 0-3° C. was introduced gradually over 1 hour. At the end of the introduction, the reaction mixture was cooled to 6-14° C. (reaction temperature was set to 9° C.) gradually over 1 hour, at the last half an hour, the pH of the solution was adjusted to 4-5 with a solution of 5N NaOH (˜4 g). The reaction temperature was kept at 6-14° C. (reaction temperature was set to 9° C.) until the end of the filtration. Solid sodium metabisulfite (2 g, 2% w/w) and toluene (140 ml) were added to the resulting solution. Adding toluene as a cosolvent prevented the sticking of the L-DOPA ethyl ester crude to the walls of the reactor during the precipitation. The operations from this stage on were done in a nitrogen atmosphere and the agitator speed was set to ˜300 rpm. A very weak stream of nitrogen was used, which produced a blanketing effect. A strong stream might have resulted in the loss of some toluene. The pH was adjusted to 6.8-7.0 with 5N NaOH solution (˜35 g) by dropwise addition over 1 hour. The solution was seeded with L-Dopa ethyl ester (1 g) and the precipitation was continued by dropwise addition of 5N NaOH solution (˜78 g) for 2 hours until pH 7.6-8.2 was reached. Precipitation started at this stage at pH 7.3, at which point the material crystallized out of the mixture. The mixture was kept at 6-14° C. for half an hour and during this period, the pH was corrected to 7.6-8.2 from time to time as necessary. The reaction mixture was kept at 6-14° C. for an additional 0.25 hours. The stirring was stopped and the batch was allowed to settle for the separation of the aqueous phase for half an hour. The lower phase was separated slowly until toluene came out of the reactor. To the content of the reactor, toluene (135 ml) was added. The precipitate was collected and washed with (3×20 ml) cold water. The mixture was cooled to less than 25° C. in order to prevent foaming before starting the azeotropic distillation. The crude wet precipitate was dried by azeotropic distillation of the water with toluene (450 ml) under vacuum until no more water was distilled out. 80 mbar vacuum was not exceeded and the jacket temperature was 50° C. and the inner temperature was 40° C. during the azeotropic distillation. At the vacuum of 80 mbar (60 mmHg), the distillation was started at Tᵢₙ=30° C. while the T_{jacket}=50° C. The crude L-Dopa ethyl ester was collected by filtration, washed with toluene (˜20 ml) and dried in a vacuum oven at 35-40° C. until a constant weight was attained. The yield of crude material was 81-83%.

Preparation of Crystalline L-DOPA Ethyl Ester

L-DOPA ethyl ester crude (50 g) and ethyl acetate which contained 0.01% BHT (w/v) (250 ml, 5 volumes relative to L-DOPA ethyl ester weight) were introduced into a 500 ml reactor. Adding toluene as a cosolvent prevented the sticking of the L-DOPA ethyl ester crude to the walls of the reactor during the precipitation. The batch was heated to 55° C. for 30 minutes and kept at this temperature until a slight turbidity remained in the solution. The hot solution was filtered through a 0.2 micron filter, washed with the remaining 20 ml ethyl acetate and returned into the reactor (the time elapsed from the beginning of the crystallization until the end of filtration did not exceed 2 hours). The clear solution was cooled to 20° C. for 2 hours (seeded at 45° C. with L-DOPA ethyl ester, at 37-38° C., massive crystallization was observed) then cooled to 5° C. for 1 hour and kept at this temperature for another hour. L-DOPA ethyl ester (cryst.) was collected by filtration, washed under nitrogen with 2×15 ml ethyl acetate which contained 0.01% BHT and dried in a vacuum oven at 35-40° C. until a constant weight was attained. The crystallization yield was 85%. The overall yield was 70%.

Purity of L-DOPA Ethyl Ester

To increase the purity of the product, additional water may be added. For example, performing the final crystallization in ethyl acetate with 1% water will result in increased purity. The amount of water to be added is easily determinable by one skilled in the art. However, it is preferable to use only ethyl acetate since the addition of water will nearly always result in loss of yield.

Levodopa ethyl ester precipitated from water has surprisingly higher purity than levodopa ethyl ester isolated via the extractive process (as performed in Milman et al.). The LDEE precipitation in water takes place at low temperatures which prevents impurities such as levodopa-levodopa ethyl ester and cyclic L-DOPA from evolving. Crystallization performed at higher temperatures (50° C.) tends to have a higher content of impurities. Therefore, the Milman process which requires extractive procedures at higher temperatures has a lower purity than the present invention.

Moreover, crude levodopa ethyl ester produced after precipitation in the subject invention may in fact have higher purity than the levodopa ethyl ester produced after crystallization for the reasons stated above. The treatment with hot (50° C.) ethyl acetate may induce increased production of impurities. However, the crystallization process is necessary for (1) controlling the particle size distribution (PSD) and (2) filtering each drug substance through a micron filter system during crystallization.

Physical Properties and Stability

L-DOPA ethyl ester as free base obtained by this process is stable, non-hygroscopic, and crystalline. The LDEE free base has a particle size from 5 to 300 micron and an average particle size of 80 micron.

The Novelties and Advantages of the Process

The Milman process comprises reacting L-DOPA with ethanol in the presence of thionyl chloride or an acid catalyst to yield crude L-DOPA ethyl ester hydrochloride. Any volatiles are then removed by vacuum distillation, the residue is then dissolved with water containing a suitable antioxidant and the pH is then adjusted to between 6.0 and 7.0 using a suitable base to yield a solution containing L-DOPA ethyl ester free base. To obtain the free base in the solvent phase, the solution is extracted with a suitable solvent such as ethyl acetate, in the presence of a suitable antioxidant. The solvent phase is then concentrated at a temperature lower than 40° C. to form a precipitate. Recrystallization of the precipitate occurs in the presence of a second suitable solvent containing a second suitable antioxidant to yield the composition of pharmaceutically acceptable, crystalline, non-hygroscopic L-DOPA ethyl ester free base.

The Milman process requires three extractions and addition of salt to the water phase at the second extraction. The addition of salt leaves the ethyl acetate saturated with salted water which necessitates two additional washings. In addition to the complications of extractions and washings, the resulting ethyl acetate contains about 7% water. Drying this ethyl acetate/L-DOPA ethyl ester solution is an involved step in the Milman process. Because most drying agents interact with L-DOPA ethyl ester, azeotropic distillation is the best route. Since azeotropic mixture of water and ethyl acetate contains a small amount of water, and since L-DOPA ethyl ester base is very sensitive to heat (producing two impurities, cyclic levodopa and levodopa-levodopa ethyl ester), vacuum distillation is required. Vacuum distillation is time consuming and the prior art process, as a whole, wastes solvent. These complications are detrimental to the resulting yield of the product. In fact, the Milman process results in only 50% yield, even though the reflux of L-DOPA with ethanol/HCl produces 96% L-DOPA ethyl ester hydrochloride in the reaction mixture. The remaining material is in the water phase in the ethyl acetate mother liquor and decomposed to L-DOPA and other byproducts during the laborious work-up.

In the process of U.S. Pat. No. 6,218,566, after removal of volatiles, the next step is to adjust the pH of the solution, add toluene and sodium metabisulfite, and then a solution of sodium hydroxide in a controlled manner (temp., stirring speed, pH, rate of addition) to precipitate L-DOPA ethyl ester free base from the aqueous phase. The L-DOPA ethyl ester is then dried by azeotropic distillation with toluene and crystallized from ethyl acetate containing BHT as an antioxidant. The azeotropic distillation step disclosed in this invention eliminates the need to use ethyl acetate for isolation of the final product as in Milman. Elimination of the drying via distillation step results in significant savings in solvents, their recovery, as well as time. L-DOPA ethyl ester is not easily extracted since it is also soluble to a certain extent in water.

Compared to the Milman process, the process of U.S. Pat. No. 6,218,566 is simpler and shorter because the capacity of production in the same reactors in terms of volume of output and yield is tripled. In the Milman process, the extraction step extracts the product into the organic phase (ethyl acetate) in a two system mixture (aqueous/organic), while in the process of U.S. Pat. No. 6,218,566, the product is precipitated from an aqueous phase. The fact that U.S. Pat. No. 6,218,566 has a crystallization step starting from a dry crude levodopa ethyl ester is important since reproducibility can be achieved, while in the Milman process, crystallization was unpredictable. Moreover, in the process of U.S. Pat. No. 6,218,566, the precipitation of L-DOPA ethyl ester is in water at an ambient temperature so that a very pure compound is obtained in greater yield than in the Milman process.

Although the process of U.S. Pat. No. 6,218,566 represents an improvement over that of Milman, the subject invention provides two further advancements.

1. Use of Toluene Instead of Water

In U.S. Pat. No. 6,218,566 and Milman et al., the removal of ethanol from levodopa ethyl ester salt was done by distillation of ethanol after the esterification reaction. The problem in this distillation step was getting rid of all the ethanol. Residual amounts of ethanol left in the distillation residue reduce the yield of precipitated levodopa ethyl ester because of the solubility of levodopa ethyl ester base in ethanol. On scale-up, this issue is more critical since it is more difficult to control the amount of residual ethanol after evaporation on large-scale production. To further reduce the amount of ethanol, water was added and another distillation was performed. The method was based on azeotropic distillation of ethanol with water.

In order to prevent the hydrolysis of levodopa ethyl ester back to levodopa after the addition of water in U.S. Pat. No. 6,218,566 and Milman et al., some precautions had to be taken. The pH of the solution needed to be adjusted to an adequate value since levodopa ethyl ester is sensitive to low pH aqueous solution, and the temperature needed to be low enough to minimize the undesired hydrolysis reaction.

The utilization of this method on large-scale production may also introduce engineering problems:

a) Long distillation times during which times the batch is subjected to relatively high temperatures;

b) The need for special equipment like vacuum pumps which are capable of acidic water distillation; and

c) In order to distill water efficiently, high vacuum is needed for which low temperature condensers should be used. In this case, the water will freeze in the condensers and may cause damage to the system.

The subject invention provides a solution to these problems by using toluene instead of water to remove the residual ethanol. The key is maintaining a nonaqueous solution until a base in water is added at step (e), which precipitates a crude L-Dopa ethyl ester free base. Toluene reduces the vapor pressure at which ethanol evaporates, which makes ethanol more volatile and hence, more easily removable. The advantages are:

a) The hydrolysis reaction is completely prevented since there is no more acidic water distilling for a long time with levodopa ethyl ester;

b) Special equipment is not needed since acidic water is not distilled; and

c) Vacuum azeotropic distillation of ethanol/toluene is advantageous since it uses lower vacuum and shorter distillation time. (The heat of evaporation of water/ethanol is higher than toluene/ethanol).

2. Separation Prior to Centrifigation

Difficulties in the filtration of the LDEE at the precipitation and crystallization stages were encountered when the first large-scale batch was produced in the manufacturing plant by the improved process of U.S. Pat. No. 6,218,566. The crude levodopa ethyl ester contained 2.7% L-Dopa. This amount was too high to be reduced to the allowed 0.5% level in the final product during the crystallization step. Not only was the amount of L-Dopa high, but also the L-Dopa particle size was very small, posing difficulties during the filtration due to blocking of the filter fabric. It was surprising that the size of the particles was very small and blocked the filter. It seems that crystallized L-DOPA has a tendency to give a small particle size distribution (PSD).

The high amount of L-Dopa was found to be due to the precipitation of L-Dopa from the aqueous phase during the long (14-17 hr) centrifugation period.

In the disclosed process of the invention, to prevent the precipitation of L-Dopa from the aqueous phase, the lower aqueous phase was separated from the product floating in the upper toluene phase before the centrifugation started. By doing this, the levodopa source was removed and the problem was solved. In order to enable this separation, an additional amount of toluene was added and the reaction mixture was then left without stirring. After one hour, the upper toluene phase contained the entire solid product floating in, it and the lower aqueous phase was discarded.

What is claimed:

1. A process for preparing a composition comprising pharmaceutically acceptable, crystalline, non-hygroscopic L-DOPA ethyl ester as free base, which process comprises:
(a) reacting L-DOPA with absolute ethanol to produce a nonaqueous solution of crude L-DOPA ethyl ester salt;
(b) removing residual volatiles, including ethanol, from the nonaqueous solution of step (a);
(c) adding toluene to the nonaqueous solution from step (b);
(d) treating the nonaqueous solution from step (c) to remove volatiles, including residual ethanol;
(e) adding a suitable base in water to the solution from step (d) under controlled conditions to precipitate a crude L-DOPA ethyl ester free base and to form an aqueous phase containing L-DOPA and an organic phase;
(f) separating the aqueous phase containing L-DOPA from step (e) from the organic phase from step (e);
(g) collecting the precipitated crude L-DOPA ethyl ester free base from step (f);
(h) drying the precipitated crude L-DOPA ethyl ester free base collected in step (g); and
(i) recrystallizing the dried, precipitated crude L-DOPA ethyl ester free base from step (h) in the presence of a suitable solvent containing an antioxidant so as to produce the composition of pharmaceutically acceptable, crystalline, non-hygroscopic L-DOPA ethyl ester free base.

2. The process of claim 1, wherein step (a) is performed in the presence of thionyl chloride, hydrogen chloride or toluene sulfonic acid.

3. The process of claim 2, wherein step (a) is performed in the presence of hydrogen chloride.

4. The process of claim 1, wherein the crude L-DOPA ethyl ester salt produced in step (a) is L-DOPA ethyl ester hydrochloride.

5. The process of claim 1, wherein the removal of residual volatiles in step (b) is effected by vacuum distillation.

6. The process of claim 5, wherein the residual volatiles removed in step (b) are ethanol and excess HCl.

7. The process of claim 1, wherein the suitable base of step (e) is sodium hydroxide or ammonium hydroxide.

8. The process of claim 7, wherein the suitable base of step (e) is sodium hydroxide.

9. The process of claim 1, wherein the addition of the suitable base in step (e) effects an adjustment in the pH of the solution to a pH range between about 3.5 and about 5.5, at which point an anitoxidant is added, and the adjustment of pH by the addition of suitable base continues to bring the pH of the solution to a pH range between about 7.6 and about 8.2 to precipitate a crude L-DOPA ethyl ester base.

10. The process of claim 9, wherein the addition of the suitable base in step (e) effects an adjustment in the pH of the solution to a pH range between 4.0 and 5.0, at which point an anitoxidant is added, and the adjustment of pH by the addition of suitable base continues to bring the pH of the solution to a pH range between about 7.6 and about 8.2 to precipitate a crude L-DOPA ethyl ester base.

11. The process of claim 1, wherein the suitable base in step (e) has been previously cooled to a temperature less than room temperature.

12. The process of claim 11, wherein the temperature is from 0 to 3° C.

13. The process of claim 1, wherein step (e) further comprises the addition of sodium metabisulphite.

14. The process of claim 1, wherein the controlled conditions from step (e) are conditions in which addition of the base solution is slowly performed in a nitrogen atmosphere, and a trace amount of L-DOPA ethyl ester is added to induce formation of precipitate.

15. The process of claim 1, further comprising adding toluene to the organic phase of step (e).

16. The process of claim 15, further comprising adding toluene to the organic phase of step (f).

17. The process of claim 1, wherein the drying of step (h) is effected by azeotropic distillation.

18. The process of claim 1, wherein the suitable solvent of step (i) is ethyl acetate, methylene chloride, or toluene.

19. The process of claim 18, wherein the suitable solvent of step (i) is ethyl acetate.

20. The process of claim 1, wherein step (e) further comprises addition of a suitable antioxidant selected from the group consisting of ascorbic acid, sodium sulfite, sodium metabisulfite, propyl gallate, and vitamin E.

21. The process of claim 20, wherein the suitable antioxidant of step (e) is sodium metabisulfite.

22. The process of claim 1, wherein the suitable antioxidant of step (i) is ascorbic acid, 2, 6-Di-tert-butyl-4-methylphenol (BHT), butylated hydroxy anisol (BHA), propyl gallate, or vitamin E.

23. The process of claim 22, wherein the antioxidant of step (i) is 2,6-Di-tert-butyl-4-methylphenol (BHT).

24. A process for preparing a composition comprising pharmaceutically acceptable, crystalline, non-hygroscopic L-DOPA ethyl ester as free base, which process comprises:
(a) reacting L-DOPA with absolute ethanol in the presence of thionyl chloride or an acid catalyst to produce a nonaqueous solution of crude L-DOPA ethyl ester salt;
(b) removing residual volatiles, including ethanol, from the solution of step (a);
(c) adding toluene to the nonaqueous solution from step (b);
(d) treating the nonaqueous solution from step (c) to remove volatiles, including residual ethanol;
(e) adding a suitable base in water to the solution from step (d) under controlled conditions to precipitate a crude L-DOPA ethyl ester free base and to form an aqueous phase containing L-DOPA and an organic phase;
(f) separating the aqueous phase containing L-DOPA from step (e) from the organic phase from step (e);
(g) adding toluene to the organic phase from step (f);
(h) collecting the precipitated L-DOPA ethyl ester free base from step (g);
(i) drying the precipitated crude L-DOPA ethyl ester free base collected in step (h); and
(j) recrystallizing the dried, precipitated crude L-DOPA ethyl ester free base from step (i) in the presence of a suitable solvent containing an antioxidant so as to produce the composition of pharmaceutically acceptable, crystalline, non-hygroscopic L-DOPA ethyl ester free base.
